(19) United States
(12) Patent Application Publication
(10) Pub. No.: US 2008/0285600 A1
(43) Pub. Date: Nov. 20, 2008

(54) Laser System for Hard Body Tissue Ablation

(75) Inventors: Marko Marincek, Ljubljana (SI); Matjaz Lukac, Ljubljana (SI)

Correspondence Address: GUDRUN E. HUCKETT DRAUDT, SCHUBERTSTR. 15A, WUPPERTAL, 42289, DE

(73) Assignee: FOTONA D.D., Ljubljana (SI)

(21) Appl. No.: 12/122,780

(22) Filed: May 19, 2008

(30) Foreign Application Priority Data
May 19, 2007 (EP) 07 010 010.2
Apr. 16, 2008 (EP) 08 007 462.8

(51) Int. Cl. H01S 3/11; H01S 3/10; A61N 5/06

(52) U.S. Cl. 372/10; 372/25; 607/89

(57) ABSTRACT

A laser system for hard body tissue ablation has a pumped laser, wherein the laser system is operated in pulsed operation with several individual pulses of a temporally limited pulse length and wherein the individual pulses follow one another with temporal pulse spacing. The pumped laser has an inversion population remaining time, the inversion population remaining time being the time within which, in the absence of pumping, the remaining inversion population of the laser energy status is reduced by 90%. The pulse spacing is in the range of ≧50 μs and ≦ to the inversion population remaining time.

### BACKGROUND OF THE INVENTION

The invention relates to a laser system wherein the laser system is adapted to be operated in pulsed operation with several individual pulses of a temporally limited pulse length and wherein the individual pulses follow one another with temporal pulse spacing.

In the field of dentistry or the like, lasers are used for removal of hard body tissues such as dental enamel, dentine or bone material. The material removal in hard tissue ablation is based on a pronounced absorption of the laser in water; despite the minimal water contents or presence of water in hard body tissue, this enables a satisfactory material removal. The laser absorption leads to local heating with sudden water evaporation that, like a micro explosion, causes material removal.

The solid-state lasers that are typically used in the field of hard tissue ablation are operated in pulsed operation as a result of their system requirements in order to avoid overheating of the laser rod. At the same time, the pulsed operation contributes to heat being generated at the treatment location only for a very short time period and within a locally limited area. However, not only the aforementioned sudden water evaporation is generated by means of the temporally limited pulse length of an individual pulse but also an undesirable heating of the surrounding tissue is caused. Moreover, at the beginning of an individual pulse a small cloud of water vapor and ablated particles is produced that shields the treatment location with regard to the temporally following section of the individual pulse and therefore reduces its effectiveness. For avoiding the aforementioned disadvantages, it is therefore desirable to use laser pulses with short pulse length, low energy, and short pulse periods as well as high repetition rate. Such a laser is known for example from US 2005/0033388 A1, with a Er:YAG laser having a pulse length of 5 to 500 fs (femtoseconds) with a pulse repetition rate of 50 kHz to 1 kHz, the latter corresponding to a pulse period of 20 μs to 1,000 μs (microseconds).

However, such an operating scheme will reduce the efficiency in other ways. A laser rod generates a laser beam only above a certain energy threshold that must be overcome by pumping, for example, by means of a flash lamp. At very short pulses of low energy, a significant portion of the pumping energy is required for overcoming this energy threshold before a usable laser energy is even made available. Therefore, according to a generally accepted teaching among persons skilled in the art, short pulses of low energy and high repetition rate have a bad efficiency and therefore provide minimal processing speed.

As a compromise, in the dental operating methods according to the prior art, as known for example from US 2003/0158544 A1, individual pulses with a pulse length of approximately 25 μs (microseconds) to 150 μs and a pulse period of approximately 16 ms (milliseconds) are used. The above described disadvantageous effects of heating the surroundings and shielding are however overcome only to an unsatisfactorily degree. The efficiency and obtainable treatment speed are minimal.

The invention has the object to further develop a laser system of the aforementioned kind in such a way that its efficiency is improved.

### SUMMARY OF THE INVENTION

This object is solved by a laser system wherein the pumped laser has a inversion population remaining time, the inversion population remaining time being the time within which, in the absence of pumping, the remaining inversion population of the laser energy status is reduced by 90% and wherein the pulse spacing is in the range of ≧50 μs and ≦ to the inversion population remaining time.

A laser system for hard body tissue ablation is proposed, comprising a pumped laser, wherein the laser system is adapted to be operated in pulsed operation with several individual pulses of a temporally limited pulse length and wherein the individual pulses follow one another in a temporal pulse period, and are separated by temporal pulse spacing. Here, the temporal pulse spacing is defined as the temporal difference between the end of one single pulse and the beginning of the next single pulse. The pumped laser has an inversion population remaining time that is the time within which in the absence of pumping the remaining inversion population of the laser energy status is reduced by 90%, i.e. to 10% of the initial value. The pulse spacing is in the range of≧50 μs, in particular ≧80 μs, and less than the inversion population remaining time.

It is important to note that the inversion population remaining time is not always equal to the spontaneous decay time of the upper laser level. For example, in laser materials with high concentration of laser active atoms or ions (such as for example Er:YAG), or with appropriately chosen additional dopants (such as for example the Cr ions in Er:Cr:YSGG), the inversion population decay process, due to the energy up-conversion processes among interacting atoms or ions, may not be exponential, and subsequently the remaining time can be significantly longer than the spontaneous decay time. The inversion population time may in such cases vary with the inversion population and thus can be determined only approximately.

In a preferred further embodiment, the laser is an Er:YAG laser with an inversion population remaining time of ≦300 μs, wherein the temporal pulse spacing is ≦300 μs.

In a preferred further embodiment, the laser is an Er:YSGG or Er:Cr:YSGG laser with an inversion population remaining time of ≦3,200 μs, wherein the temporal pulse spacing is ≦3,200 μs.

In a preferred further embodiment, the laser is a solid-state laser with an inversion population remaining time of ≦200 μs, wherein the temporal pulse spacing is ≦200 μs.

With these time values, the invention deviates from the afore described teachings of the prior art and is based on the following recognition:

When an ablative laser light pulse is directed onto the hard tissue, ablation of the tissue starts and leads to the emission of ablated particles above the hard tissue surface, forming a debris cloud. The debris cloud does not develop instantaneously. Particles begin to be emitted after some delay following the onset of a laser pulse, after which they spread at a certain speed and within certain spatial angle above the ablated tissue surface. So in the beginning the emitted particles are close to the surface, and at longer treatment times the particles are well above the surface. The debris cloud interferes with the laser beam, resulting in laser light scattering. The undesired scattered portion of the laser beam is present to a significant extent only at the later time steps of the single laser pulse.

From a scattering viewpoint, the temporal pulse spacing between two subsequent single pulses should be longer than the time the debris cloud needs to settle down, the longer the better. This way there is no debris cloud remaining from the previous pulse. In particular, when between the end of one single pulse and the beginning of the next single pulse there remains sufficient time, which time is greater than the cloud decay time of approximately 90 microseconds, any subsequent laser pulse will not encounter a debris cloud remaining from the preceding laser pulse.

However, from the viewpoint of laser efficiency it is advantageous to not use temporal pulse spacing that is as long as possible. This is because there is some inversion population of the laser energy status remaining after the end of the laser pulse. When a laser material is supplied with energy by pumping, the individual atoms are successively moved into the higher laser-enabling energy state. A significant share of the atoms remains at this higher energy state for a short period of time even after termination of the pumping process and even after termination of the laser emission. This period of time is limited by the inversion population remaining time, being the time within which in the absence of pumping the remaining inversion population of the laser energy status is reduced to 10% of the initial value. In case pumping for the second pulse starts early enough the threshold is reduced as the laser has been already pre-pumped from the previous pump pulse. From this viewpoint, the temporal pulse spacing should be shorter than the inversion population remaining time, i.e. the time within which, in the absence of pumping, the remaining inversion population of the laser energy status is reduced to 10%. The shortening of the pulse spacing in accordance with the present invention utilizes this effect in that after termination of a very short individual pulse and after completion of the very short temporal pulse spacing within the inversion population remaining time, there is still residual energy in the laser material that is available for the subsequent individual pulse.

So, a compromise is found according to the invention, where the temporal pulse spacing should be longer than the cloud decay time and shorter than the inversion population remaining time as follows: The residual laser energy is found to be useful to a technical extent for temporal pulse spacing ≦ to the inversion population remaining time. For suitable pulse lengths between 10 and 120 microseconds the cloud decay time is approximately on the order of 50 microseconds, so in the inventive combination the pulse spacing is between including 50 microseconds, in particular 80 microseconds and including the inversion population remaining time.

Contrary to the prior art prejudice, the laser can be operated with the afore defined short temporal pulse spacing, and in consequence with short pulse lengths being shorter than the pulse period, at low energy and at high efficiency so that a high treatment speed is enabled. The pulse period and thus the temporal pulse spacing between two individual pulses is large enough so that a debris cloud of removed material, water, and water vapor can escape from the beam path. The pulse length of the individual pulses is short enough that the shielding effect of the water vapor generation caused in the first time period of the individual pulse is of reduced importance or is even negligible during the subsequent second time period of the individual pulse. The impairment of the laser beam by the removed material is minimized in the second period of the individual pulse; the absorption of the laser light is reduced. The scattering of the beam is minimized so that the removal precision is improved. The heat load of the tissue surrounding the treatment location is minimized.

In a preferred further embodiment, the pulse length is in the range of ≧10 μs and ≦120 μs. For this preferred range, it is important to note that scattering of the light in the debris cloud represents a problem only when the cloud is high enough above the surface so that it can scatter the light of the laser beam considerably away from the original laser beam size. Since typical beam sizes are within 0.1 and 2 mm, scattering becomes a serious problem when the cloud reaches a height of approximately 2 mm or higher. This happens approximately within 90 to 110 microseconds after the laser pulse onset. It therefore has been found, that with laser pulse durations of approximately 120 microseconds or shorter, the effect of scattering is almost non-existent, compared to the case when pulse duration of approximately 400 microseconds is used.

From the scattering viewpoint the pulse duration should therefore be equal to or shorter than 100 microseconds, the shorter the better. However, in regard to technical considerations (achievable pump powers with diodes that cannot pump enough energy within short times; or, when flash lamp pumping is used, exponentially decreasing flash lamp lifetimes at shorter pulse durations) it is desirable to have long pulse lengths. Therefore a suitable compromise is provided when applying pump pulses with a duration ≦120 microseconds and ≧10 microseconds.

However, even at this range of pulse lengths the laser efficiency of lasers such as Er:YAG, Er:YSGG or Er:Cr:YSGG is reduced as their operating efficiency is better in case longer pulse durations with higher energy outputs and lower repetition rates are used. However, by choosing the inventive train of pulses with temporal pulse spacings within the inventive range, some of the efficiency is regained that is lost by using shorter pulse durations, respectively, shorter pulse lengths. So, it is the combination of both pulse spacing and pulse duration ranges, that makes laser efficiency high enough even at the reduced light scattering.

In a preferred further embodiment, the individual pulses are combined to pulse sets that follow one another in a temporal set period wherein the pulse sets each comprise at least three individual pulses. Expediently, the pulse sets each have maximally 20 individual pulses, preferably however eight to twelve and in particular ten individual pulses. The temporal set period is preferably ≦50 ms, advantageously ≦30 ms and in particular approximately 20 ms. In the aforementioned embodiment, the individual pulses, known in the prior art, are at least partially replaced by the inventive pulse sets. Maintaining the aforementioned upper limit of the number of individual pulses per pulse set avoids overheating of the laser rod. Between the individual pulse sets there is enough time for cooling of the laser rod. The aforementioned minimum number of individual pulses per pulse set leads to an effective utilization of the residual energy that remains in the laser material after pumping so that the arrangement as a whole can be operated with high efficiency.

### BRIEF DESCRIPTION OF THE DRAWINGS

One embodiment of the invention will be explained in the following with the aid of the drawing in more detail. It is shown in:

FIG. 1 a schematic illustration of a debris cloud generation during the course of a single laser pulse resulting in scattering of the laser beam;

FIG. 2 a diagrammatic illustration of the temporal debris cloud development close to the treated surface;

FIG. 3 a diagrammatic illustration of the temporal debris cloud development at a greater distance to the treated surface compared to FIG. 2;

FIG. 4 a diagrammatic illustration of the debris cloud time delay dependence on the distance from the ablated surface;

FIG. 5 a diagrammatic illustration of the temporal course of pulse sets according to the present invention;

FIG. 6 an enlarged diagrammatic illustration of a detail of a pulse set according to FIG. 5 with the temporal course of the individual pulses;

FIG. 7 a measuring diagram of the actual pulse course according to FIG. 6 for explaining the utilization of the energy that is stored within the laser rod.

### DESCRIPTION OF PREFERRED EMBODIMENTS

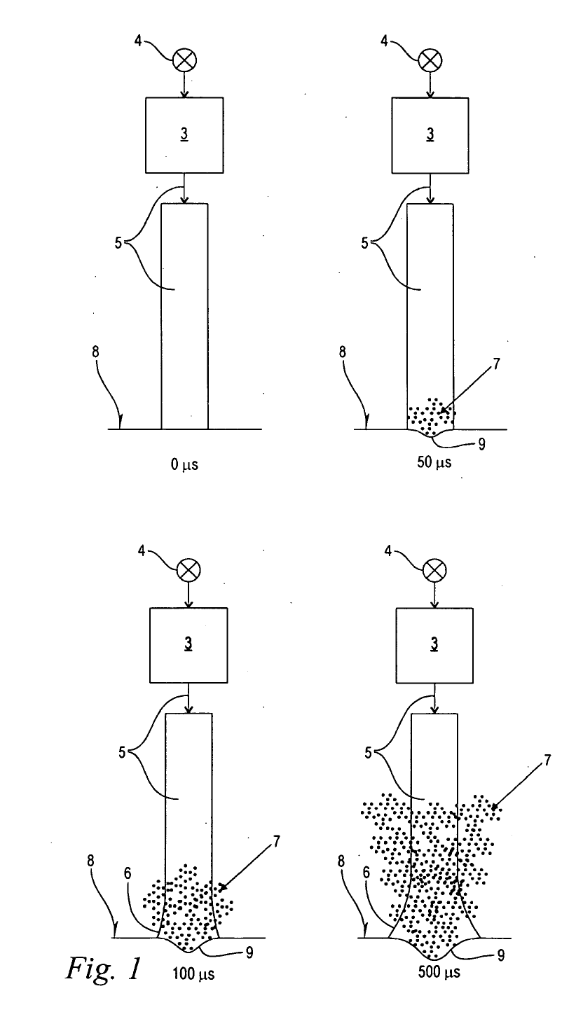

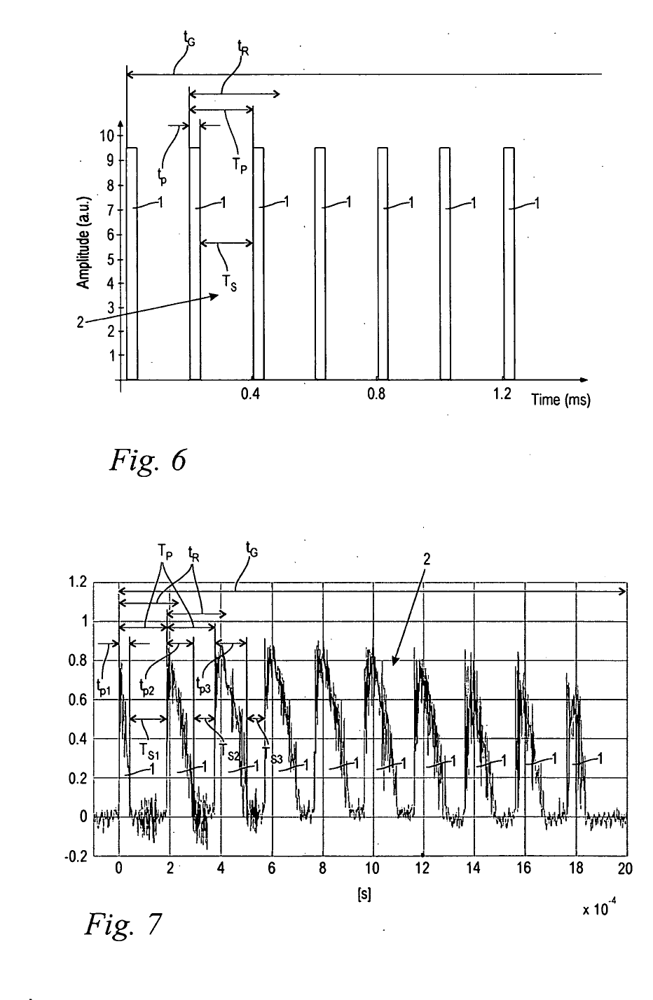

FIG. 1 shows a schematic illustration of a debris cloud 7 generated during the course of a single laser pulse at four different points of time, namely at the beginning of the single laser pulse at 0 μs, followed by subsequent time steps of 50 μs, 100 μs and 500 μs. A laser system comprises a laser 3 that is a solid state laser and is pumped by a flash lamp 4. The laser is a solid-state laser with an inversion population remaining time t_{R} of ≦200 μs, as illustrated in FIGS. 6, 7. The inversion population remaining time t_{R} is the time within which in the absence of pumping the remaining inversion population of the laser energy status is reduced by 90%. The laser is preferably an Er:YAG with an inversion population remaining time t_{R} of ≦300 μs, or an Er:YSGG (or Er Cr:YSGG) laser with an inversion population remaining time t_{R} of ≦3200 μs (FIG. 7). However, other solid state lasers or any other type of lasers such as liquid, diode, gas or fiber lasers can be used.

During pumping by the flash lamp 4, the laser 3 generates a pulsed laser beam 5, each pulse of the laser beam 5 corresponding to the flash lamp pulse. The pulsed laser beam 5 is directed to a treated surface 8 of hard body tissue like dental enamel, dentin or bone material. The laser beam 5 is schematically depicted as an arrow close to the laser 3. However, in practical use the laser has a specific diameter in the range of 0.1 to 2.0 mm, as shown close to the treated surface 8. Note that other pumping mechanisms, such as diode pumping and other methods not mentioned but well known in the art, may be applied instead of pumping with flash lamps.

When the ablative laser light pulse is directed onto the hard tissue, ablation of the tissue starts and an ablation area 9 is formed; this leads to the emission of ablated particles above the hard tissue surface 8, forming a debris cloud 7. The debris cloud 7 does not develop instantaneously, as can be seen in FIG. 1 for the time value of 0 μs. Particles begin to be emitted after some delay following the onset of the laser pulse, after which they spread at a certain speed and within certain spatial angle above the ablated tissue surface. In the beginning the emitted particles are close to the surface, and with increasing time the particles are well above the surface, as can be seen in FIG. 1 for the time intervals of 50 μs, 100 μs and 500 μs. The debris cloud 7 interferes with the laser beam 5, resulting in laser light scattering and a scattered portion 6 of the laser beam 5. The undesired scattered portion 6 is present to a significant extent only at the later time intervals of the single laser pulse, as can be seen e.g. for the time interval of 500 μs.

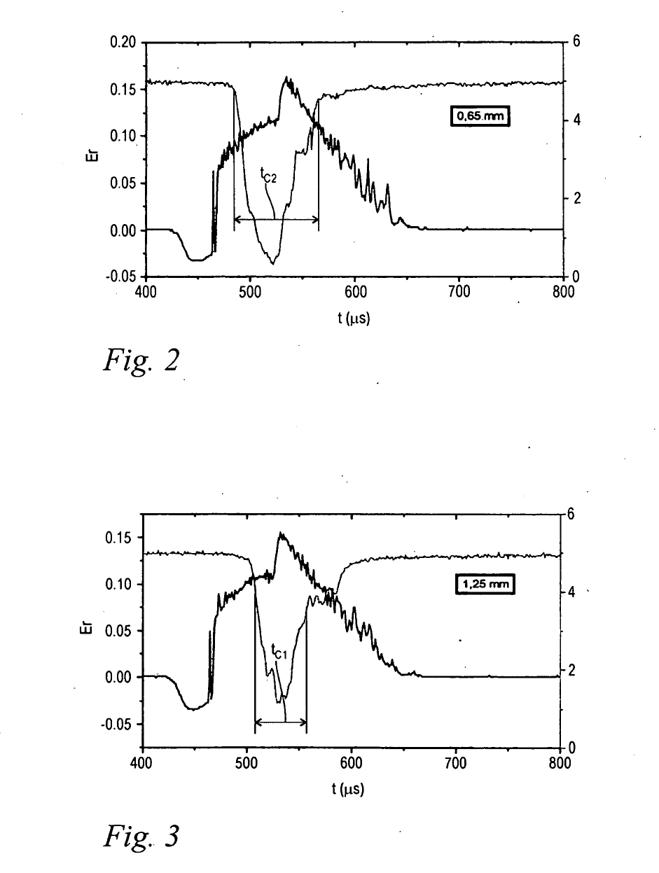

As an example, FIGS. 2 and 3 show the cloud development at the distances 0.65 mm and 1.25 mm from the treated surface 8 (FIG. 1) respectively. In the FIGS. 2 and 3 the upper curve represents the laser pulse temporal shape and the lower curve represents the amount of scattered light at a particular spatial distance from the ablated surface. As can be seen, the cloud development, measured by the level of light scattering, occurs later at larger distances from the surface 8 (FIG. 1). It can be seen from the scattered light curves, that the debris cloud 7 (FIG. 1) has typical cloud decay times t_{C1}, t_{C2} of approximately 50 μs and 80 μs respectively. Within the cloud decay times t_{C1}, t_{C2} the debris cloud 7 has settled down to an extent, that it does not disturb the laser beam 5 (FIG. 1) significantly, and that it does not generate a significant scattered portion 6 of the laser beam 5 (FIG. 1). Pulse spacings T_{S}, as described infra in connection with FIG. 5 to 7, are therefore chosen to be equal to or longer than the cloud decay times t_{C1}, t_{C2}, i.e. ≧50 μs, and in particular ≧80 μs, in order to allow time for the debris cloud 7 (FIG. 1) to settle down between individual pulses.

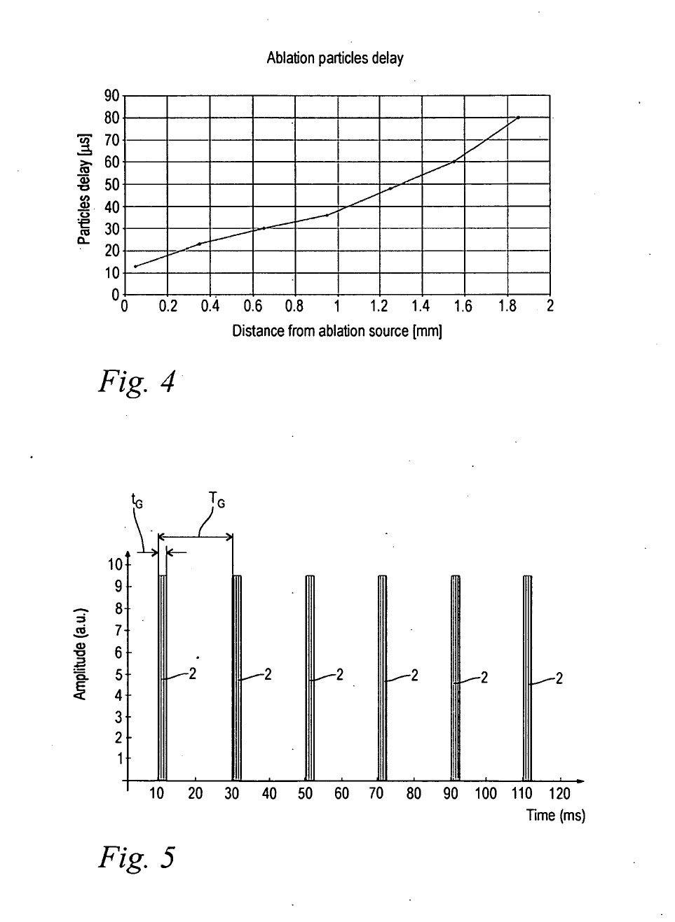

FIG. 4 shows the dependence of the time delay of the cloud development on the distance from the treated surface 8. It is important to note that scattering of the light in the debris cloud 7 (FIG. 1) represents a problem only when the cloud is high enough above the surface so that it can scatter the light of the laser beam 5 (FIG. 1) considerably away from the original laser beam size. Since typical beam sizes are within 0.1 and 2 mm of diameter, scattering becomes a serious problem when the cloud reaches a height of approximately 2 mm or higher. This happens (see FIG. 4) approximately within 90 to 110 microseconds after the laser pulse onset. It therefore has been found, that with laser pulse durations of approximately 120 microseconds or shorter, the effect of scattering is almost non-existent, compared to the case when pulse duration of approximately 400 microseconds is used.

Referring now simultaneously to FIGS. 1 to 7, the inventive laser system is adapted to be operated for hard body tissue ablation, the laser 3 being operated in pulsed operation wherein individual pulses 1 (FIG. 6) of the laser 3 or of a laser beam generated by the laser 3 are combined to pulse sets 2 as explained infra in connection with FIGS. 6 and 7.

FIG. 5 shows in a schematic diagram the temporal course of the pulse sets 2 according to the invention. In this connection, the course of the amplitude of the pulse sets 2 is illustrated as a function of time. The pulse sets 2 follow one another in a temporal set period T_{G}. The temporal set period T_{G} is expediently ≧50 ms, advantageously ≦30 ms, and is in the illustrated embodiment of the inventive method approximately 20 ms. The individual pulse sets 2 have a temporal set length t_{G} of, for example, approximately 2 ms. Depending on the number of individual pulses 1 provided infra the value of the temporal set length t_{G} can vary.

FIG. 6 shows an enlarged detail illustration of the diagram according to FIG. 5 in the area of an individual pulse set 2. Each pulse set 2 has at least three and maximally 20 individual pulses 1, respectively; preferably, each pulse set 2 has eight to twelve individual pulses 1 and in the illustrated embodiment according to FIG. 7 there are ten individual pulses 1 of which, for ease of illustration, only seven individual pulses 1 are illustrated in FIG. 6. The individual pulses 1 have a temporal pulse length tₚ and follow one another in a temporal pulse period T_{P}, the temporal pulse period T_{P} being the time period from the beginning of one single pulse 1 to the beginning of the next, subsequent pulse 1. The individual pulses 1 follow one another with a temporal pulse spacing T_{S}, the temporal pulse spacing T_{S} being the temporal difference between the end of one single pulse 1 and the beginning of the next single pulse 1. For generating the individual pulses 1 of the laser beam, the laser 3 is pumped by means of the flash lamp 4 (FIG. 1) in pulsed operation. The temporal course of the flash lamp pulses corresponds with regard to the pulse length tₚ, the pulse spacing T_{S}, the pulse period Tₚ, and the pulse set period T_{G} to the temporal course of the individual pulses 1 or of the pulse sets 2 of the laser beam. In FIG. 6, the amplitude of the laser beam or of its individual pulses 1 is schematically plotted as a function of time wherein the temporal course of the individual pulses 1, for ease of illustration, are shown as rectangular pulses. In practice, the pulse course deviates in accordance with the illustration of FIG. 7 from the schematically shown rectangular shape of FIG. 6.

The pulse spacing T_{S} is, according to the invention, in the range between 50 μs, in particular 80 μs, and the inversion population remaining time t_{R}. For the particular case of an Er:YAG laser the pulse spacing T_{S} is ≦300 μs. For the particular alternative case of an Er:YSGG laser the pulse spacing T_{S} is ≦3,200 μs. Preferably, for a solid state laser the pulse spacing T_{S} is ≦200 μs. The pulse length tₚ is in the range of ≧10 μs and ≦120 μs, in particular ≦50 μs. The pulse period T_{P} is chosen as an example to be 200 μs. However, different pulse periods T_{P} may be applied. With the pulse length tₚ in the range of ≧10 μs and ≦120 μs, and the sum of one pulse length tₚ and one pulse spacing T_{S} being equal to one pulse period T_{P}, the actual pulse spacings T_{S} are in the range of ≧80 μs and ≦190 μs.

FIG. 7 shows a measuring diagram of the course of an actual laser pulse wherein an Er:YAG laser was pumped with flash pulses of constant pulse length tₚ and constant pulse period Tₚ of 200 μs in accordance with the illustration of FIG. 6. The illustrated pulse set 2 has a total of ten individual pulses 1; in this connection, the amplitude of the generating laser beam is illustrated as a function of time. It can be seen that the first individual pulse has approximately a triangular course with a pulse length tₚ₁ of approximately 50 μs. After the first pulse a pulse spacing Tₛ₁ of approximately 150 μs has lapsed, the second individual pulse 1 with a pulse length tₚ₂ of approximately 100 μs, slightly increased in comparison to the pulse length of the first individual pulse 1, follows wherein the second individual pulse 1 relative to the first individual pulse 1 has a more “filled-out” course with a higher total energy quantity. The second pulse 1 with the pulse length tₚ₂ is followed by a pulse spacing T_{S2} of approximately 100 μs. For an unchanged energy input by means of pulsed pumping in accordance with the course of FIG. 6, approximately after the third individual pulse 1 a pulse length tₚ₃ of approximately 120 μs will result, followed by a pulse spacing Tₛ₃ of approximately 80 μs. The third individual pulse 1 has, in comparison to the first two individual pulses 1, a more filled-out pulse course so that the energy of the individual pulse 1 is further increased relative to the preceding first two individual pulses 1, in spite of the pump energy remaining the same. The short pulse spacings Tₛ₁, Tₛ₂, Tₛ₃ of approximately 150 μs to 80 μs are well below the inversion population remaining time t_{R} of ≦300 μs of the Er:YAG laser as used here. In consequence, during the pumping process in particular of the first two individual pulses 1, a portion of the pumped energy is saved in the laser rod and is not completely given off in the form of laser energy. As a result of the short pulse spacing Tₛ, a part of the saved energy is available for generating the energy yield in the case of the subsequent individual pulses 1.

On the other hand, as can be further derived from FIG. 7, the shown pulse spacings Tₛ are ≧50 μs and even ≧80 μs. Between the end of one single pulse 1 and the beginning of the next single pulse 1 there remains some time in the range between including 80 μs and including 150 μs, which is in the same order or even greater than the cloud decay time t_{C} of approximately 50 μs or 80 μs (FIG. 2, FIG. 3). So any subsequent laser pulse will not encounter a debris cloud 7 (FIG. 1) remaining from the preceding laser pulse 1.

Toward the end of the pulse set 2, i.e., upon completion of, for example, ten individual pulses 1 with a temporal set length t_{G} of approximately 2 ms, the energy of individual pulses 1 is reduced as a result of thermal effects. After lapse of the set period T_{G} (FIG. 5) and the start of a new pulse set 2, the complete energy schematic according to FIG. 7 is available again, however.

The specification incorporates by reference the entire disclosure of European priority documents 07 010 010.2 having a filing date of May 19, 2007 and 08 007 462.8 having a filing date of Apr. 16, 2008.

While specific embodiments of the invention have been shown and described in detail to illustrate the inventive principles, it will be understood that the invention may be embodied otherwise without departing from such principles.

What is claimed is:

1. Laser system for hard body tissue ablation, the laser system comprising a pumped laser, wherein the laser system is adapted to be operated in pulsed operation with several individual pulses of a temporally limited pulse length and wherein the individual pulses follow one another with a temporal pulse spacing, wherein the pumped laser has an inversion population remaining time, the inversion population remaining time being the time within which in the absence of pumping the remaining inversion population of the laser energy status is reduced by 90% and wherein the pulse spacing is in the range of ≧50 μs and ≦ to the inversion population remaining time.

2. Laser system according to claim 1, wherein the pulse spacing is ≧80 μs.

3. Laser system according to claim 1, wherein the laser is an Er:YAG laser with an inversion population remaining time of ≦300 μs and wherein the pulse spacing is ≦300 μs.

4. Laser system according to claim 1, wherein the laser is an Er:YSGG or an Er:Cr:YSGG laser and has an inversion population remaining time of ≦3,200 μs, wherein the pulse spacing is ≦3,200 μs.

5. Laser system according to claim 1, wherein the laser is a solid-state laser with an inversion population remaining time of ≦200 μs and wherein the pulse spacing (T_{S}) is ≦200 μs.

6. Laser system according to claim 1, wherein the pulse length is in the range of ≧10 μs to ≦120 μs.

7. Laser system according to claim 1, wherein the individual pulses are combined to pulse sets following one another in a temporal set period, wherein the pulse sets each comprise at least three individual pulses.

8. Laser system according to claim 7, wherein the pulse sets each comprise maximally twenty individual pulses.

9. Laser system according to claim 7, wherein the pulse sets each comprise eight individual pulses to twelve individual pulses.

10. Laser system according to claim 7, wherein the pulse sets each comprise ten individual pulses.

11. Laser system according to claim 7, wherein the temporal set period is ≦50 ms.

12. Laser system according to claim 7, wherein the temporal set period is ≦30 ms.

13. Laser system according to claim 7, wherein the temporal set period is approximately 20 ms.
